(19) United States
(12) Patent Application Publication
(10) Pub. No.: US 2017/0072283 A1
(43) Pub. Date: Mar. 16, 2017

(54) SPORTING DEVICE AND WEARABLE COMPUTER INTERACTION

(71) Applicant: RUSSELL BRANDS , LLC, Bowling Green, KY (US)

(72) Inventors: Mark Joseph Davisson, Rensslaer, IN (US); Michael J. Crowley, Attleboro, MA (US); Kevin King, Dublin, OH (US); Matthew Anthony Tyson, Columbus, OH (US)

(73) Assignee: RUSSELL BRANDS , LLC, Bowling Green, KY (US)

(21) Appl. No.: 15/121,316

(22) PCT Filed: Feb. 25, 2015
(86) PCT No.: PCT/US2015/017505

§ 371 Date: Aug. 24, 2016

Related U.S. Application Data
(60) Provisional application No. 61/946,497, filed on Feb. 28, 2014.

(51) Int. Cl. A63B 71/06; G09B 19/00; A63B 69/00; A63B 24/00; G10L 15/22; A61B 5/00; H04N 5/765; H04N 5/272; G06K 9/00; A63B 43/00; A61B 5/11

(52) U.S. Cl. CPC A63B 71/0622; A63B 43/00; G09B 19/0038; A63B 69/0071; A63B 24/0062; A61B 5/1118; A61B 5/6803; A61B 5/6802; A61B 5/6895; H04N 5/765; H04N 5/272; G06K 9/00671; G10L 15/22; A63B 2225/50; A63B 2220/803; A63B 2071/0666; A63B 2220/806; A63B 2220/833

(57) ABSTRACT

A computer-implemented method is disclosed that includes capturing data about motion of a sports object caused by one or more athletes manipulating the sports object, transmitting the captured data out of the sports object wirelessly in real time while the one or more athletes are still manipulating the sports object, and presenting information that incorporates the captured data about motion of the sports objects with one or more wearable devices.

### CROSS-REFERENCE TO RELATED APPLICATIONS

This application claims the benefit of priority to U.S. Application No. 61/946,497 filed Feb. 28, 2014. The disclosure of the prior application is considered part of and is incorporated by reference in the disclosure of this application.

### TECHNICAL FIELD

This document relates to interaction between a sporting device and wearable computers.

### BACKGROUND

Sports are big business. Owners of teams and athletes who play professionally earn very high incomes. The Olympics create great national pride every two years. Collegiate athletics provide a base around which alumni can rally, and provide student athletes with opportunities to further their educations while developing their skills. And lower-level high-school and recreational athletics provide further mechanisms for athletes to have fun, mature, and learn.

People who put such an importance on sports, and those who want to get better at sports also go to extreme efforts to improve their performance. Some attend special camps taught by experts, at great expense. Others pay for court time or field time or ice time, and travel long distances for games and tournaments. Yet others hire coaches for expensive one-on-one training sessions. Much of the coaching and evaluation that occurs in such activities is subjective and prone to personal biases. At the same time, such extensive human involvement drives up the costs for an athlete who wants to learn what he or she does well or does poorly, and who wants to improve.

### SUMMARY

This document describes systems and techniques that may be used by an athlete or by spectators to more conveniently be presented with real-time information about current athletic performance by the athlete and potentially other athletes. The information can be presented on a head-mounted display worn by such people, including on a display that overlays, on a natural view for the user, an electronic display device that superimposes information about the athletic performance. For example, a player dribbling a basketball or a player who has just released a shot may be provided feedback regarding the quality of their gameplaying in a manner that can allow them to improve their gameplaying, such as feedback that indicates an unduly low arc on a just-taken jump shot, so that they adjust and improve the arc on their next shot attempt. Such information can be generated from data that is transmitted out of the basketball (or other sporting device such as soccer ball, baseball, hockey puck, golf club or ball, etc.) from a motion-sensing assembly in the ball, where the motion-sensing assembly has previously established a communication session that a computer that feeds the head-mounted display.

Video can also be captured by the device worn by the athlete and may have superimposed on it information generated from the data captured by sensors in the ball and other information. Such video may be later reviewed by the player to better understand what he or she did right or wrong during a playing session. The video may also be viewed in real-time by a remote coach, who may provide audible feedback through an earpiece of the head-mounted device and/or via text or graphics provided to the head-mounted display of the device.

In other implementations, the information can be overlaid on views for people other than the playing athletes or coaches. For example, spectators who are wearing devices like Google Glass at a basketball game or soccer match may have information shown in real-time in coordination with the current game play on the displays of their glasses, including information derived from sensors inside a game ball or other sporting device. As just two examples, a numeric value of g-force applied to a ball by a kick or slam dunk may be displayed, or a curve representing an arc of a shot may be displayed, including being displayed over or adjacent to a video replay of a recent scoring play. Such numeric and/or graphical representation may allow those spectators to obtain additional information about the play, and thereby improve their enjoyment of the game without distracting unduly from their live viewing of the game.

In one implementation, a computer-implemented method is disclosed that comprises capturing data about motion of a sports object caused by one or more athletes manipulating the sports object; transmitting the captured data out of the sports object wirelessly in real time while the one or more athletes are still manipulating the sports object; and presenting information that incorporates the captured data about motion of the sports objects with one or more wearable devices. The sports object can be a sports ball and the data can be capturing by one or more motion sensors mounted inside the sports ball. The wearable devices can be worn by the one or more athletes and the presented information can provide them with feedback about their athletic performance with the sports ball. In addition, the wearable devices can comprise electronic glasses having a visual display that shows the presented information.

In certain aspects, the electronic glasses are connected to a computer and wireless receiver arranged to obtain data from a wireless transmitter inside the sports ball in real-time. Also, the electronic glasses can present to an athlete currently manipulating the sports ball a numerical indication of their manipulation of the sports ball. The method can also include automatically wirelessly connecting the wireless receiver and wireless transmitter upon sensing that the wireless receiver and wireless transmitter are near each other. The method can also include recording a video that comprises video captured by the electronic glasses overlaid with information derived from the captured data about motion of the sports ball. In addition, the method can include determining whether the sports ball is undergoing dribbling actions or is undergoing shooting actions, and selecting a type of information to be displayed based on the determination, wherein: If the sports ball is determined to be undergoing dribbling actions, the presented information indicates a quality of the dribbling across multiple dribbles; and If the sports ball is determined to be undergoing shooting action, the presented information comprises indications of arc, accuracy, or both for a current shot.

In some aspects, the wearable devices comprise electronic glasses worn by a plurality of spectators watching the plurality of athletes in person, and the presented information annotates action in a game that the plurality of athletes are currently playing. Moreover, the method may comprise recognizing a verbal command spoken by one of the one or more athletes, and determining a mechanism to use in analyzing data from the sports item in response to content of the verbal command. In other aspects, the method includes communicating between worn computing devices of different ones of the one or more athletes to share information from the sports device, or presenting with the wearable devices and to one of the one or more athletes, instructions for improving athletic performance for the one of the one or more athletes, using the captured data.

In another implementation, a computer-implemented system is disclosed that comprises a wearable computing device having a head-mounted display that is capable of superimposing a display of data over a field of view of a user wearing the head-mounted display; one or more programs on one or more tangible recordable media of the wearable computing device having instructions that when executed, perform operations including: (a) receiving data about motion of a an athlete-manipulated sports object transmitted from a computing device inside the athlete-manipulated sports object, and (b) presenting information via the head-mounted display information that incorporates the captured data about motion of the athlete-manipulated sports object.

In certain implementations, such systems and techniques may provide one or more advantages. For example, athletes can be presented in real-time with information without having to distract themselves from their gameplay. As just one example, a player may be provided with information about a game while the game is still occurring, such as clock and score data, or can be provided with performance data while practicing, such as data about how fast or hard the player is dribbling a basketball, or an angle at which a shot left the athlete's hand or passed through a hoop. As other examples, spectators can be shown information that improves their watching of a game, whether they are attending the game in person or are watching on a television screen. For example, real-time data and statistics can be displayed on glasses worn by certain spectators to show the spectators basic scoring information that is otherwise displayed on a scoreboard in an arena (so they do not have to take their attention away from the gameplay) or supplemental information, such as the g-force on a slam dunk, the amount of curvature on a scoring soccer kick, and the like. In certain implementations, sports teams or partners may provide extra information in this manner as a paid supplemental service for certain fans, and can derive additional revenue from such services. Also, advertising or other promotional material may be presented with such heads-up-presented information, so that additional revenue can be derived from providing such additional useful information.

Other features and advantages will be apparent from the description and drawings, and from the claims.

### DESCRIPTION OF DRAWINGS

FIG. 1A shows an individual athlete equipped with a head-mounted display.

FIG. 1B shows spectators at a basketball game equipped with head-mounted displays.

FIG. 2A is a view from an individual athlete equipped with a head-mounted display.

FIG. 2B is a view from a spectator equipped with a head-mounted display.

FIG. 3A is a schematic diagram for a head-mounted display and a sporting ball sensor package.

FIG. 3B is a schematic diagram for a portable device that can communicate with a head-mounted display and a sensor package.

FIG. 4 is a flow chart of a process for capturing sports data and presenting it via a head-mounted device.

FIG. 5 is a block diagram of an illustrative system for collecting, presenting, and storing data from a sporting event.

FIG. 6 shows a general computer system that can provide interactivity with data regarding a sporting event.

### DETAILED DESCRIPTION

In general, this disclosure relates to mechanisms by which data captured by sensors in a gaming device can be processed and presented on wearable computing devices, such as head-mounted displays in a form similar to the GOOGLE GLASS product from Google Inc. of Mountain View, Calif. Generally described here are “see through” displays that allow a user to have an almost full field of vision of things that are occurring in front of the user, and to have that view annotated with text, graphics, animations, and video that the user looks through to see their natural field of view, and/or that are presented in an unobtrusive area of the user's field of view, such as in a corner of the field of view that the user can quickly check and then return their view to the main action in front of them.

Disclosed are systems and techniques for presenting information about an athletic event, such as a practice session or a game. Two general examples of such systems are discussed here, in the form of (a) wearable computing devices for an athlete or (b) wearable computing devices for a spectator of an event. An athlete wearing such a computing device may be provided with immediate feedback to help the athlete improve his or her performance in a sport. For example, a basketball player may be provided with information received from a sensor package inside the basketball that measures objective aspects of dribbling, passing, and shooting of the basketball by the athlete and by other players on the court. As just one example, as soon as an athlete releases a basketball shot, information may be displayed in a corner of the athlete's field of view that indicates the quality of the shot, such as information about the rotation of the ball in the shot, the angle of release and angle of entry for the shot, and the speed with which the athlete prepared for and released the shot measured in fractions of a second or seconds, and a speed of the ball (e.g., in feet per second) at the point of release.

A similar wearable computing device may be provided to a coach of the athlete during a practice session or during a game. The coach may see the same information or same type of information as the athlete sees, and may use such information to determine what sort of instructions to provide to the athlete or to a team. For example, in a training session, a coach may use his natural field of view to observe how a player takes a practice shot, and as soon as the shot is released may look into the corner of his field of view to obtain digital information measured by a sensor package in the ball. The coach may then combine his observations of the two types of information in providing immediate feedback to the player about the shot. For example, the coach may observe that the player had a hiccup in the way that she raised the ball for a shot, and may have also seen that the computed time of release for the shot was objectively too long. In such a situation, the combination of information for the coach may indicate that the coach needs to instruct the player in improving her motion in raising the ball to take a shot.

Spectators such as attendees at a basketball game may be presented with yet other forms of information including information from data obtained by a sensor package in the basketball or other sporting device. For example, attendees of a basketball game in an arena may have their natural field of view (watching the game) annotated by a heads-up display with information about a game, including information from sources other than a sensor package in the ball, information derived from a sensor package in the ball, and a combination of the two. For example, clock, score, foul, and player data may be displayed at appropriate points during a game, such as the clock information being shown at all times, and foul or scoring player information being shown immediately after the corresponding player commits a foul or makes or misses a score. Such information about a current happening in the game may be displayed for a predetermined period of time, such as several seconds, so as to permit the spectator to digest the information, and then may be replaced with other information, such as how television broadcasts provide temporary overlays of particularly relevant information and then remove it. Information from the ball may include information like that in the example for the player discussed above in addition to other information that may not be helpful as feedback to a player, but may be entertaining for a spectator, such as information about g-forces on a slam dunk, distance of curvature on a pitch or a soccer goal, and other similar information.

Such gathered information may be obtained by the head-mounted device directly from the sensor package in the ball; from a short range intermediary such as a mobile device carried by the athlete, where the sensor package communicate with the mobile device and the mobile device communicates with the head-mounted display; or by more long distance techniques such as by way of a local area network and wide area network. For example, for fans watching a basketball game, information from a sensor package in the ball may be reported to a central server system along with statistics information for the particular game and statistics information across a wide range of events such as season information for the players in a basketball game. The data from the ball may come from a LAN, while data about other games may arrive via a WAN. Each of the types of information may be combined into an electronic transcript for the game, where each track in the electronic transcript represents a category of common data, such as motion data from the ball, scoring data (e.g., entered by a human scorer at a game or watching the game remotely), other statistical data, and video from one or more cameras captured during the game. Each of the tracks may be coordinated according to a common clock or clocks, where the common clock may include one or more of a normal time of day clock, a remaining time game clock, or an arbitrary timing mechanism to provide a baseline for coordinating (timewise) the various types of data and determining at what point in time events leading to the data occurred.

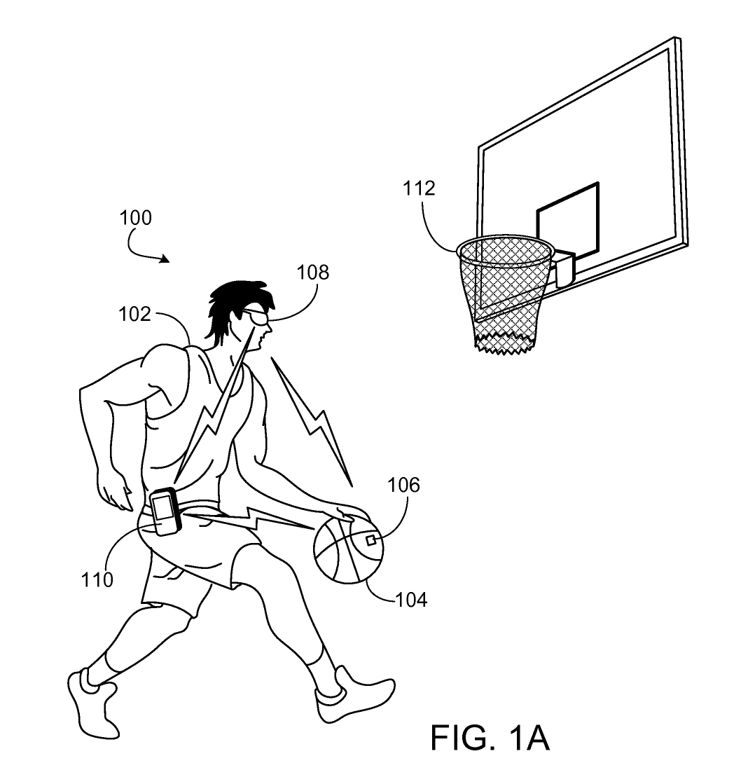

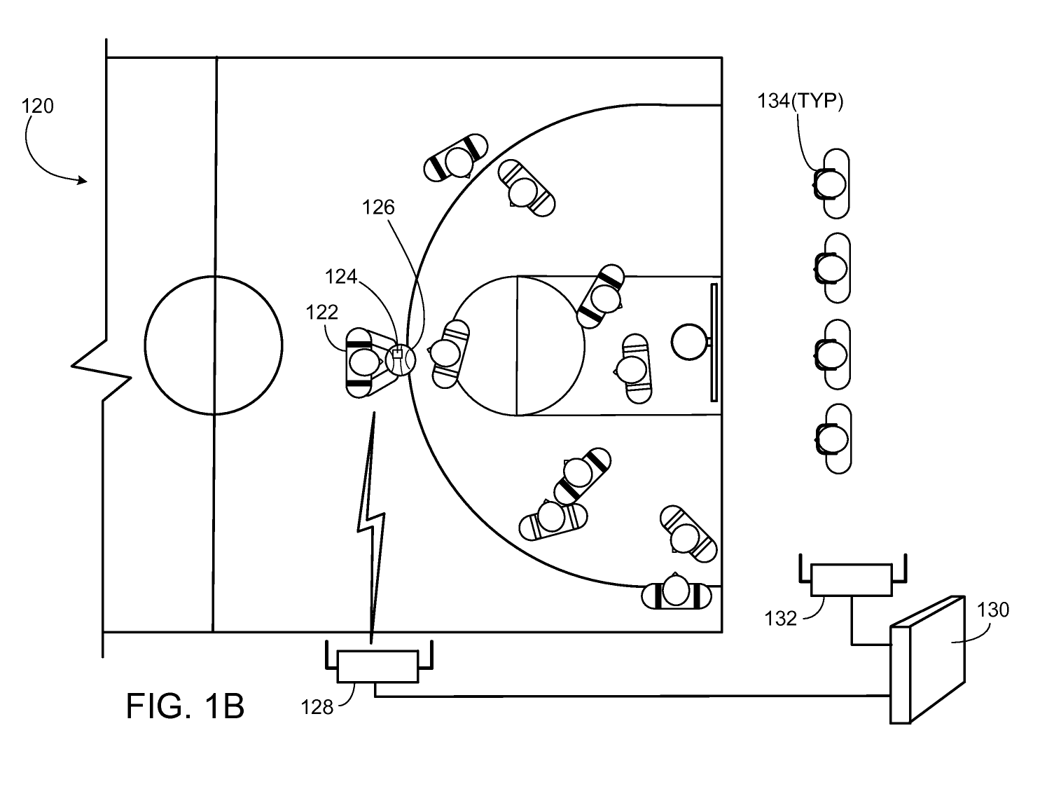

FIG. 1A shows an individual athlete 102 equipped with a head-mounted display. In the setting 100 shown in the figure, the athlete 102 is taking part in a game or a training session and is wearing a heads-up display 108 such as a pair of GOOGLE GLASS devices. The heads-up display 108 may include a mechanism for visually annotating text and graphics into a corner of a field of view of the athlete 102. For example, the heads-up display 108 may include a pair of glasses that have a projection or other display mechanism mounted in an upper corner of the field of view so that the athlete 102 may remain undistracted while looking straight forward, but may choose to look upward and into a corner of the field of view in order to see the additional annotation information. The heads-up display 108 may also include additional mechanisms, including a microprocessor (and associated chip set); related memory storing applications and data for operating the heads-up display 108; wireless connectivity features; and an inertial sensor package that can measure force, acceleration, direction, angle of inclination, and other appropriate motion data.

The athlete 102 is seen in the figure dribbling a basketball 104 that has mounted within it a sensor package 106. The athlete 102 may be preparing to pick up the basketball 104 to shoot it through a basketball hoop 112. In some implementations, the basketball hoop 112 may also be instrumented such as by including sensors in or around it to detect when a basketball has contacted the backboard or the hoop 112, or has gone through the hoop 112 so as to register a made or missed basket.

The sensor package 106 may include a set of inertial sensors that may be purchased off the shelf for use in devices such as smart phones and other devices where measurements of direction, speed, acceleration, position, and angle relative to ground are desirable. The sensor package 106 may also include preprocessing circuitry, including an appropriate microprocessor with associated memories storing instructions for processing the raw data coming from the sensors in the sensor package 106. For example, the processing system may be programmed to identify particular motions that have been taken with respect to the ball such as dribbles, passes, and shots, and to process the received data to characterize those actions. For example, the processing system may create derived information from the sensor data to indicate an angle of a shot and angle of entry of the shot through a basketball hoop, g-forces applied to dribbles, shot, or dunks, time period that it takes an athlete to perform certain actions where the processing components determine the beginning and ending point of the actions from the captured motion data, and other derived information that may be useful to obtain from the basketball 104.

The sensor package 106 may then use wireless communication circuitry to transmit the raw data, the derived data, or both to sources outside of the basketball 104. For example, as shown by lightning bolts in the figure, a short range wireless connection may be made between the sensor package 106 and the heads-up display 108, another device 110 worn by the athlete 102, or both.

The components with which the sensor package 106 communicates may depend on the particular needs of a particular implementation. For example, where the heads-up display 108 is full-featured and includes complex computing capabilities, the device 110 may not be needed, and communication can be directly between the sensor package 106 and the heads-up display 108. Alternatively, when additional computing power or interaction is needed, the device 110 may provide such computing power and then transmit additional derived information such as simple display information to the heads-up display 108. As one example, the device 110 may use data transmitted from the sensor package 106 to generate a graphical representation regarding some aspect of the performance by athlete 102. That graphical presentation may then be transmitted as a simple bitmap to the heads-up display 108.

The device 110 may also include extended communication capabilities not available with heads-up display 108. For example, heads-up display 108 may include only short range wireless communication capabilities, such as Bluetooth. The device 110 may include longer-range communication capabilities such as WiFi or cellular data connections so as to be able to provide data from the sensor package 106 more broadly or to bring in additional data in generating a display for heads-up display 108. For example, sensor package 106 may generate data about a particular athlete's performance, and device 110 may access a remote database over the Internet using cellular data connections to obtain information about performance by other athletes, so that heads-up display 108 may present a comparative performance display for athlete 102 that compares the performance by athlete 102 to recorded performance from other athletes at different times and at locations remote form the athlete 102.

In other implementations, device 110 may be provided to display or otherwise present information that is supplemental to that that is displayed or provided by heads-up display 108. For example, the resolution of display 108 or its size may be inferior to that provided by device 110, so that low resolution data may be provided immediately and conveniently on heads-up display 108, and additional high-resolution data may be provided by device 110 after the player has stopped a drill or other performance and removed device 110 to look at it. As one example, device 110 can store forward-looking video captured by heads-up display 108, and can play back that video along with data that represents forces on the basketball 104 that were occurring at the corresponding time when the particular frames of the video were captured.

Typically, the wearing of such a heads-up device 108 by an athlete while performing an athletic activity would occur during a practice session (as opposed to a game), with the heads-up device 108 providing feedback to the athlete 102 in order to help the athlete 102 better appreciate the quality of their athletic performance and allow the athlete 102 to immediately take steps to improve that performance. The feedback may be objective, in that is derived from data from sensor package 106, and is not biased by the subjectivity of a particular human observer. The data may also be derived in relatively complex manners, use data from third-party sources that are remote from the current location of the athlete 102, and be presented in various complex manners, including with color graphics, animations, and video either alone or overlaid with graphics and animations. As just one example, a digital camera may be located off to the side of athlete 102 when the athlete 102 is practicing in a shooting drill, and may capture a wide angle view of the athlete shooting a ball. That camera may be in communication with the heads-up display 108 or device 110. When the athlete takes a shot, the sensor package 106 may obtain a data about the shot and the camera may, when the shot is completed, extract several seconds of video that shows the shot and may pass the video file to the device 110 or the heads-up display 108 automatically. The receiving computing device may then annotate the video, such as by displaying an drawn arc over the path of the ball and providing visual numerical data adjacent to the arc such as to indicate angle of a shot, release time for the shot, rotation of the shot, and other information. The annotated video may then be provided for display on the head-up display 108, including automatic display that is not requested by the athlete 102. In such a manner, athlete 102 may take a shot, and while getting positioned to obtain a rebound, may immediately see a video replay (e.g., in a corner of his or her field of view) of the shot from the side to better see what the athlete's form looks like so that he or she can improve that form. The athlete 102 may then take another shot and the cycle of data collection, transformation, and combination may repeat so that the athlete 102 may see whether changes he or she may make in his or her form cause an improvement in result. The athlete may continually repeat the process without having to provide any instructions to the devices in between shots, and this cycle may improve the athlete's 102 performance.

FIG. 1B shows spectators at a basketball game equipped with head-mounted head-up displays. In general, in this example, the heads-up displays are provided to spectators of a basketball game such as heads-up display 134. Again, the particular example is basketball, and in this situation it involves an actual game with a full contingent of players from two teams. Other sports may also implement similar data collection and presentation on heads-up displays.

As with the prior example, a basketball 124 is provided with a sensor package 126 inside of it that may have wireless communication capabilities for getting data acquired by the sensor package regarding motion of the basketball 124 to computing resources located external to the basketball 124. The sort of data and processing performed by sensor package 126 may be similar to that discussed with respect to FIG. 1A, but as described more fully here, the data may be used in different manners for presentation to spectators than it would for presentation to an athlete.

Also, when the data is presented to different individuals essentially simultaneously, particular ones of those individuals may see the same data as other ones of the spectators, or different data, depending on a class in which they are identified or according to customization parameters that they are provided. For example, at the game shown here, spectators such as spectator 134 may be shown one type of data, whereas a coach for one of the teams may be shown a different type of data. In particular, spectators may be shown data that is expected to provide entertainment value, whereas coaches may be provided with data about their particular team that provides informational value, such as data that may indicate that one of their players is slowing down and tiring and bus should be removed from the game for a time. The difference in data provided may be the result of the two types of viewers using different data presentation applications loaded on their displays, or by having different parameters entered into a similar application. In this manner, the presentation of supplementing sports data may be user-specific and customized.

In the figure, a basketball half-court 120 is shown and a point guard 122 is holding the basketball 124. The point guard 122 may be preparing to take a three-point shot with the basketball 124, to pass the ball 124, or to dribble with the ball 124.

As the ball 124 is moved, a sensor package 126 may generate motion and related data of a variety of forms, and processing circuitry in the sensor package 126 may create derived data from it. The data may be streaming essentially constantly out of the ball 124 and may be picked up by one or more wireless transceivers 128 that may be proximate to the basketball court 120. Such transceivers 128 may be dedicated to collecting data from the basketball 124 and sensor package 126, and may be operated as a network that is separate from other networks in the facility, including by operating at a frequency that is distinct from other networks such as a WiFi network. The data may also be encrypted or otherwise protected from interference by nefarious parties within the facility so as to maintain the quality of the data.

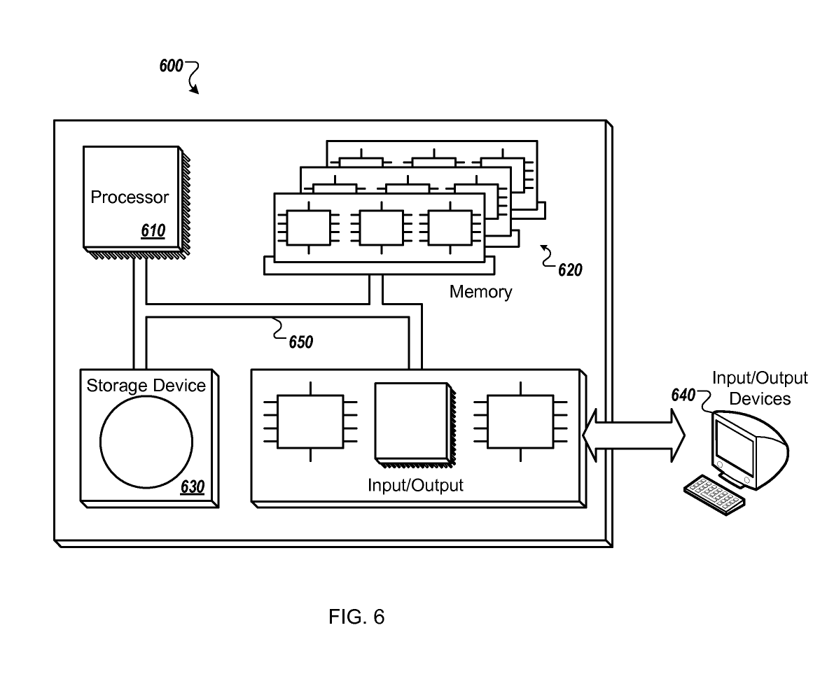

The transceiver 128 can interact, either directly or indirectly, with a server system 130 that may perform a variety of functions, and that is in turn connected to a transceiver 132. Transceiver 132 may be a WiFi access point that operates according to known protocols (e.g., IEEE) so that mass-market head-mounted displays like display 134 may communicate with the transceiver 132. In a typical arena, multiple such transceivers 132 may be distributed so that fans throughout the arena may wear their personally-owned head-mounted displays to a game and have information about the game provided on the displays. Particular types of information that can be displayed to spectators is discussed in more detail below with respect to FIG. 6.

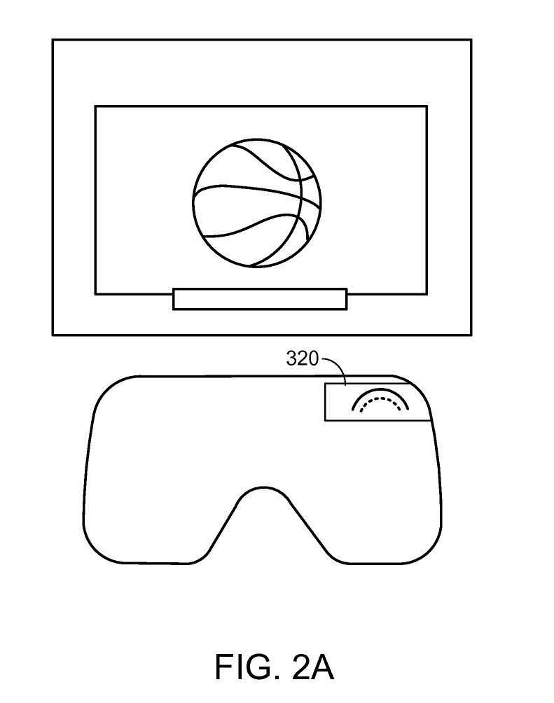

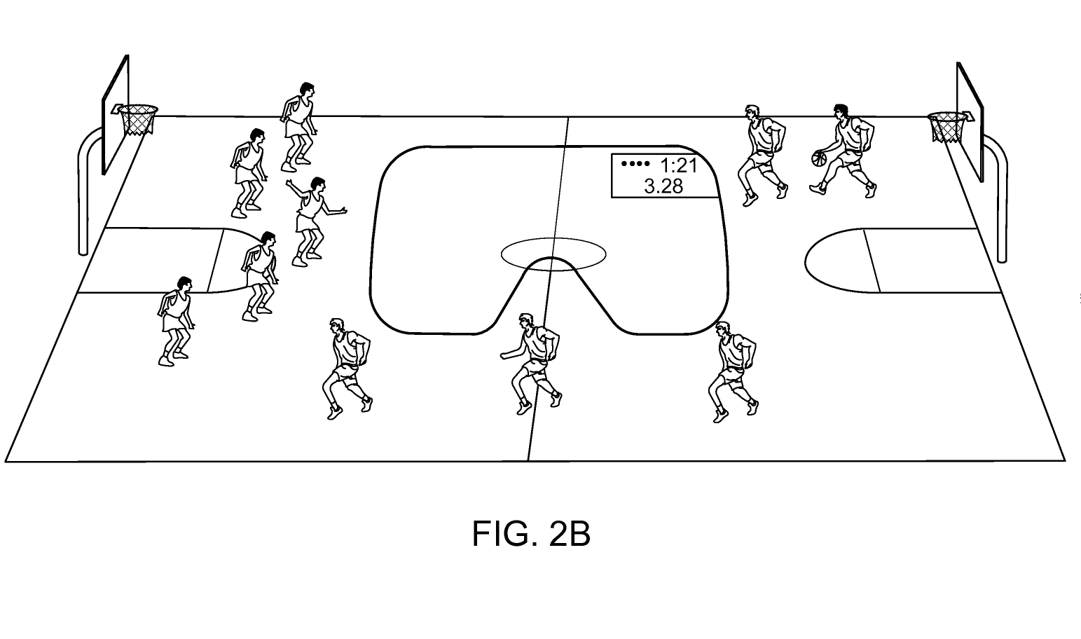

FIG. 2A is a view from an individual athlete equipped with a head-mounted display. The view here has been simplified to remove the head of the athlete and to show the background that would be part of the athlete's field of view, in addition to electronic annotations provided by a heads-up display attached to or as part of glasses worn by the athlete.

In this example, the athlete is looking at a basketball hoop on a basketball court. A shot recently-released by the athlete is about to pass through the hoop. In the time since the athlete released the shot, a sensor package in the basketball captured initial motion data, processed that data to recognize the raising of the ball into a shooting position and a release of the shot, and obtained initial data about the shot, such as the shot release angle relative to horizontal (and perhaps a computed angle that the shot will be at when it falls back to 10′ and is presumably at rim height (and hopefully passing through the hoop)). In the time while the ball was aloft, such data was also communicated out of the ball and to the heads-up display, and a textual and graphical representation was produced for display (either by the in-ball electronics or by the heads-up display electronics, or a combination of the two)

As can be shown in the upper-right corner of the field of view through the glasses, a display shows the angle of release for the shot (32°) and a graphical arc showing the actual arc (e.g., in solid line) adjacent an optimal arc (e.g., in dashed line). Thus, the athlete may take the shot and then briefly glance up and to the right to immediately see the data for the shot, then quickly run to get the rebound and shoot again, after mentally taking into account the feedback from the data for the shot.

In other situations, the athlete may perform other drills with the basketball and the heads-up display may automatically display data appropriate to the actions taken by the athlete based on determinations made by the system about the type of actions being taken. For example, when the athlete is dribbling, the heads-up display may show information relevant to dribbling, such dribbling speed and an indication of the control the athlete has over the dribble. When the athlete pulls up for a shot, the type of display information may change automatically to information about release times and speeds, shot arc, and shot rotation.

Other sorts of information that the athlete (or a coach) may want to see during a practice or a game include physiological data about the athlete. For example, the athlete may wear a separate sensor pack that may acquire ECG and other data in real-time about the athlete. The heads-up display may then show the athlete's current pulse, blood pressure, ECG recording, and similar physiological data.

Video may also be captured by a front-facing camera in the glasses worn by the athlete. Such video may be superimposed with the information shown in the heads-up display and potentially with additional information. Such super-position may place the information in a rectangle in the upper-right corner of a screen so as to simulate the view that the athlete had of the scene. The information may also be placed over other parts of the scene or even placed to the side of the video. For example, a player may review his practice on a laptop computer having a split screen, where video captured by this glasses is shown in one part of the display and a variety of collected data is shown in another part. The athlete may select various controls on the display to pause, fast-forward, and reverse the video or to select video form other practices or games, and to show different types of data or show additional detail about certain data (e.g., expanding a display of player heart rate to show a graph of heart rate over time).

FIG. 2B is a view from a spectator equipped with a head-mounted display. The glasses in this example are a similar retro style to those in FIG. 2A, but the shape and style could take a variety of forms, and could be fitted with no lenses, non-corrective lenses, or corrective lenses directed to a prescription for the particular user. Again, the glasses are supplemented with a heads-up display that may be made from a layer applied to the surface of the glasses, may be projected onto the lenses of the glasses, or may be generated by or projected onto an item that extends in front of the lenses, such as by hooking around from the right side of the frames for the glasses.

In this example, a full court is shown behind and through the glasses (though in actuality, a user's field of view through the glasses would be much greater than shown here because the spectator's eyes would be closer to the glasses than the viewpoint in this figure), as the glasses are worn by a spectator who has mid-court seats near the top of the lower level in an arena. A player has just completed a dunk to the right-hand basket, and the ball is being brought upcourt while the players set at the left side of the court. While the ball was (and is) being handled by players, a sensor package in the ball is constantly collecting data and determining, based on signatures of the data, what sort of operations are being performed with the ball, such as dribbling, passing, and shooting (either jump shot, hook shot, or dunk). The data is also nearly constantly being streamed out of the ball to courtside transceivers and then provided to a server system that further processes the data.

As a result, the heads-up display is able to combine data from the basketball sensor package showing the g-force applied to the ball when it was recently dunked, and also data form other sources such as four dots to indicate that the game is in the fourth quarter, and a time remaining in the quarter. In this example, the display of g-force may be provided for only a few seconds, so that the spectator can look at it quickly out of the corner of her eye if she wants. The data may then be replaced with other temporary data, such as the system determining which player currently possesses the ball or which player made (or missed) the most recent shot, and showing game data for that player—where the display may change each time the ball changes hands. Possession of the ball may be determined automatically, such as by beacons worn by each player that are sensed by electronics in the ball (so that the player closest to the ball is registered as possessing the ball), or by cameras mounted around and above the court whose feeds are provided to object recognition systems to identify player uniform numbers and visual characteristics in identifying which player currently has the ball.

The heads-up display of the figure shows traditional scoring data (quarter and time) and textual and graphical data derived from in-ball sensors. As noted above and below, additional information may be displayed and may be further derived from data like that displayed. For example, comparative data may be displayed, showing, e.g., the top 5 dunks of the season in terms of g-force, or the top 5 for the player who just dunked. Combination of force data and game data may also be displayed, such as graphs correlating a certain player's force for dribbling or speed of dribbling as a function of clock time, a function of how many minutes they have played in a game, or a function of how many continuous minutes they have been on the court. Such combined and derived data may be based on separate sources of currently-streaming data, saved data from the particular game, saved data from other games by the same team (which may be accessed from a database managed by the team), and saved data from games involving other teams (which may be accessed from a database managed by a third party, such as the league, or a third-party data bureau).

A variety of other forms of data may be captured, and a variety of other forms of information may be presented to a wearer of a head-mounted display like the glasses shown here. For example, images captured by a camera of the heads-up display may by subject to object recognition to identify sporting devices, and motion data may be gather using such analysis in addition to motion sensors in the ball, or as an alternative to using motion sensors in the ball. When the motion information is captured in such a manner, the same sort of information displays may be generated as discussed above and below. Object recognition may also be used for other purposes, such as by changing the appearance (e.g. color, size, background, traces through the air, etc.) on the heads-up display, particularly when the augmented portion of the display covers substantially all of the user's field of view rather than just a small corner of the field of view. For example, if the shot is in the optimal range for shot arc, the background of the ball can be colored green as it is being displayed in the heads-up view finder.

In another example, player recognition of individuals may be employed using images captured by the heads-up display (e.g. facial recognition, bar code, QR code or other machine-readable code on jersey, number on jersey, etc.). Historical information about the other player(s) may then be displayed on the heads-up display, including information about how they have interacted with an instrumented sporting device. For example, such functionality could be used to join sessions between different players interacting live or across the Internet. The functionality may also be used by a coach to display historical information about basketball shooting and dribbling stats from a central database that gathers motion data and other data about performance by various athletes.

As another example, the heads-up display may receive voice commands to start sessions with instrumented sporting devices such as starting specific activities, drills, games, real-time remote competitions, etc. Voice commands may also be used to switch between drills. For example, an athlete can say “figure 8 dribble drill,” and the heads-up display and sensor package may coordinate to begin capturing, analyzing, and presenting data and information for such a drill. The user may speak “done,” when he or she wants to have the information about the drill presented to him or her. These voice commands can also be used to interact in real-time with historical data gathered from the sensor sporting devices. For example, an athlete can speak “Ok Glass—Start InfoMotion game of horse with Kevin King,” and the system will identify Kevin King in a contact list for the speaking athlete, obtain data about Mr. King and the athlete, including data about prior games of horse between the two, and present relevant information as the game goes on (e.g., score of the current game and odds of one player winning or losing based on prior performance in horse between the two).

Moreover, an application may gather data and video form various activities by a player in order to automatically produce a coaching session for the player. As one example, the player may request a coaching session on three-point jump shots. The system may then gather video and data from games and practice sessions in which the player shot three-point shots, and may also gather data from other players to serve as benchmarks—either players who have data similar to the requesting athlete or those who are considered to be exemplars of three-point shooting proficiency. The system may then allow the athlete to readily access video and data across all of his own three-point attempts and see data correlated to made versus missed shots. The athlete may also watch third-person-view video of the made or missed shots next to or overlaid with similar video of the athlete's other shots (missed or made), or to other athletes, so as to better identify hitches in the shot deliver that need to be corrected.

Coaches may also interact readily with their heads-up displays to mark a coaching session for later review (and the third-person-view video discussed here may be captured by the coach's heads-up display). For example, a video may be overlaid (e.g. recorded/overlay video viewed in real-time or reviewed off-line) via coach gestures and other ways to document in the video good behaviors and suggested improvements for the activities being performed while using an instrumented sporting device. For example, a coach could tap the side of the heads-up display while watching a player perform a drill to set a bookmark that can later be found easily, to add audio comments on player technique, and/or other gestures or voice commands to augment the video to focus on elbow flair, lack of legs in shots, and similar comments. In other instances, instructions from a coach may be provided as text to an athlete in real time or later, where the text may be typed by the coach or initially spoken by the coach and then converted, particularly when the coach is at a location remote from the athlete.

In addition to athlete, coaches, and spectators, augmented views may be provided to officials during a game, such as an instant replay view by which a referee could quickly determine (without leaving the playing field) whether a player was in or out of bounds, whether a play was completed before time ran out, etc. Such referee-related information may also include time of impact from an instrumented sporting device with game time synchronized video, Network Time Protocol synchronization of multiple devices across the Internet, and other similar actions.

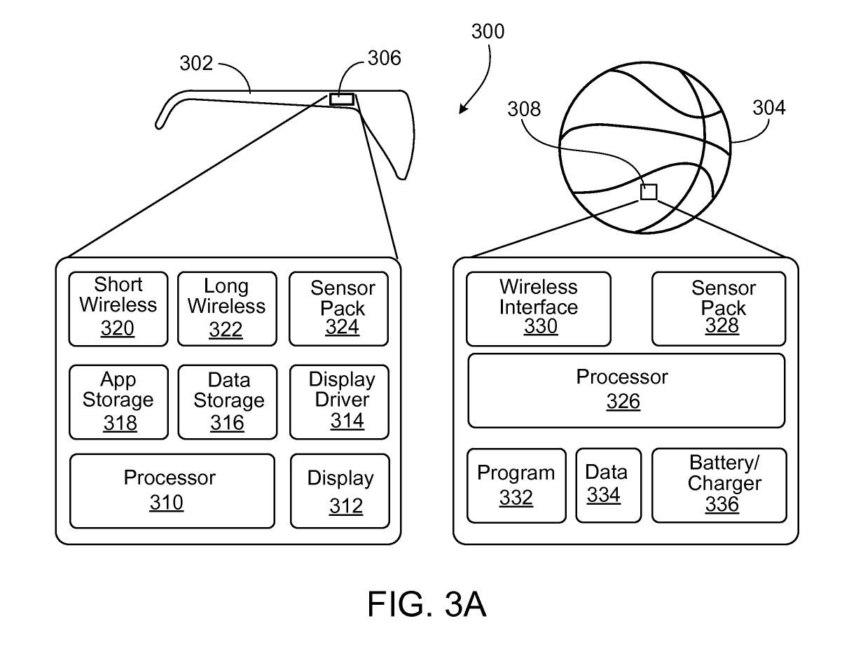

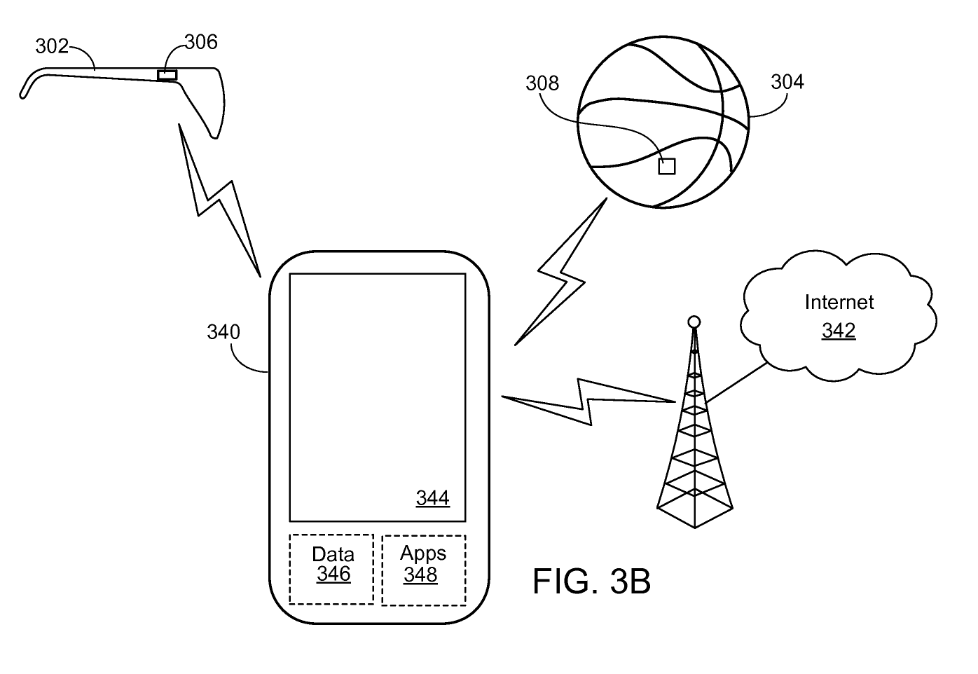

FIG. 3A is a schematic diagram for a head-mounted display and a sporting ball sensor package. In general, the device is shown here make it a system 300 by which motion data for a sporting device may be captured, processed, and presented by way of visual, auditory, or tactile presentation to one or more individuals wearing headmounted presentation devices, such as electronic glasses that provide an overlay of information on a natural of a user who is wearing the glasses. In the discussion about the components of system 300, computing activities may be carried out by way of specialized circuitry, software or firmware operating on a general or specialized microprocessor, a combination of the two, or in other appropriate manners.

The system 300 includes a heads up display 302 and a sporting device 304 in the form of a basketball. The sporting device 304 may be handled by one or more athletes and capture information about how it is handled. The heads-up display 302 may present information derived from such captured data and from other acquired data.

Referring now more specifically to the heads-up display 302, a processing system 306 may be mounted in a housing of the display 302 or otherwise in communication with mechanisms or providing presentation of information on the display 302. The processing system 306 may include a number of components for obtaining information and presenting such information to a wearer of the heads-up display 302. For example, a sensor pack 324 may be included in the heads-up display 302 to capture information about motion by a person who wears the heads-up display 302. The sensor pack 324 may include inertial sensors, such as accelerometers, gyroscopes, and similar sensors that may be obtained commercially in pre-manufactured packages, so as to identify a direction that a user of the heads-up display 302 is facing, an angle of inclination of the head of such user, and motion by the head of the user. In addition, sensor pack 324 may include GPS functionality to determine an absolute location of the user within acceptable ranges of uncertainty.

The processing system 306 also includes a short wireless interface 320 and a long wireless interface 312. The short wireless interface 320 may take the form of a Bluetooth or WiFi interface, and may be configured to receive and provide data to other communicating devices in close proximity to the heads-up display 302, such as WiFi access points nearby, and to/from sporting device 304. The long wireless interface may communicate over longer distances, such as with cell towers as part of a cellular data access plan that provides general Internet connectivity.

In certain implementations, the short wireless interface 320 and the long wireless interface 312 may be used substantially simultaneously for different purposes. For example, the short wireless interface 320 may be used to obtain motion data from sporting device 304, and the long wireless interface 312 may be used to obtain information over a network such as the Internet, including information about other uses of sporting devices similar to sporting device 304, so that heads-up display 302 can provide comparative information (between the current user of the device 304 and the other users) for a wearer of the device.

Processor 310 may be used to provide processing operations on the heads-up display 302, and may access applications storage 318 and data storage 316 in doing so. Applications storage 318 may hold any of a number of different applications that a user of the device 302 has downloaded to the device 302. Such applications may be acquired from an online app store, such as the APPLE ITUNES store operated by Apple Corporation of Cupertino, Calif. Data storage 316 may take a familiar form and may include volatile or nonvolatile storage that holds data regarding the user's interaction with sporting device 304, including raw motion data and derived data that is created at least in part from the raw motion data. The data storage 316 may also include video files and other similar data to be used in generating presentations by the device 302.

A display 312 and associated display driver 314 may be used to generate visual displays on the device 302. The display 312 may take a variety of forms, including a display that occupies a small portion of a user's field of view, such as with the GOOGLE GLASS system, or a display that is overlaid on a substantial portion of a user's field of view, either for one or eye or for both eyes of the user. Generally, the display 312 will be provided so as not to substantially interfere with the user's natural field of view through the heads-up device, view of actual, non-computer-generated, scenes in front of the user. In this respect, the heads up display 302 is different than immersive goggles that provide a user's entire field of view as computer-generated, and block substantially all of the user's natural field of view in front of the user.

Referring now to sporting device 304, there is located inside sporting device 304 a sensor package 308. The sensor package 308 is generally configured to sense motion and other aspects of a manner in which the sporting device 304 is handled, to perform processing on such sensed data, and to wirelessly transmit the derived data through the bladder or outer skin of the sport being device 304 to an external device such as heads-up display 302.

To perform such wireless transmission of data, the sensor package 308 includes a wireless interface 330. Such interface 330 may take a standard form and be implemented by commercially available chip sets, such as communicating by Bluetooth, ZigBee, or WiFi standards. The interface 330 may both receive information, such as in the form of commands to obtain and provide data from the device 304, and also transmit data, such as to get motion data out of the sporting device 304 and to a location where it can be further processed and presented to a user.

The wireless interface 330 may communicate with a microprocessor 326 which may also take a variety of familiar and commercially available forms. The microprocessor 326 may in turn communicate with program storage 332 and data storage 334. The program storage 332 may store instructions for capturing motion data and for processing such data when a program in program storage 332 is loaded by processor 326. The data storage 334 may store motion data and other derived data for a period of time until such data is transmitted out of the sporting device 304 by wireless interface 330. For example, data storage 334 may be established to store data in a first in first out (FIFO) manner so as to serve as a form of buffer for data to be output by the sporting device 304.

A sensor pack 328 is provided and may take a familiar commercial form that includes accelerometers and other familiar mechanisms for measuring motion data. The processor 326, under the control of program code, may obtain such data from the sensor pack 328 in a raw form that is general to any application that may be made of the data, and may convert the raw data to a derived form that is specific to analysis of sporting actions taken with the sporting device 304. For example, the raw data may take the form of three-axis accelerometer data and other similar data, and the processor 324 may analyze such data to determine what sport actions were taken, times at which particular sporting actions occurred (started and ended), such as dribbles, passes or shots, and may further derive the data to identify parameters of those dribbles, passes, or shots (e.g., the force of the actions, the curve on the ball from such actions, etc.).

As one example, the processor 326 may generate a data structure that characterizes the number of dribbles that a particular player has taken during one handling of the sporting device 304, the g-force applied to each of the dribbles, and the amount of time between each dribble, such as measured from hand contact to hand contact or from for floor contact to floor contact. In this manner, the sensor package 308 may perform much of the processing that is needed to convert the raw motion data to data that is readily usable and displayable to an athlete or other person.

Battery/charger 306 36 is provided in sporting device 324 to power the other electronic components in sporting device 324. The battery may provide a consistent source of energy for the other components, and the charger may be provided to recharge the battery when it has been depleted. The charger may take the form of an inductive charging system that uses a coil in the shell of the device 304 to receive power from a coil external to the device 304 when the device 304 is placed on a charging surface.

FIG. 3B is a schematic diagram for a portable device 340 that can communicate with a head-mounted display and a sensor package. As shown in the figure, portable device 340 may serve as an intermediary between sporting device 304 and heads-up display 302, and also may serve as an intermediary between either of those two devices in a wide area network that may include the Internet 342 and various services for accepting and providing information through the Internet 342. The device 340 may take the form of a tablet computer, a smart phone computer, and other various known devices they may provide computing functionality and also permit the operation of custom programs on such devices. The portable device 340, in this example, includes a visual display 344 to provide visual output for a user (e.g, a touch sensitive LED or LCD display), in addition to a speaker for audible output, and mechanisms for providing haptic output such as vibrations of the device 344.

In typical implementations, the device 344 may communicate wirelessly through the Internet 342 (via a cell carrier system) and have various different commercial applications 348 loaded into memory on it for execution on a microprocessor of the device 340. In some implementations, such applications may be downloaded from a commercial app store that permits downloading of a variety of applications from different publishers, and may include applications for accepting, processing, and presenting data related to activity in sporting events, including data derived from sporting device 304. For example, a program for presenting statistics relating to one or more athletes handling a basketball may be downloaded by people who have purchased sporting device 304, which may serve as a convenient mechanism by which the manufacturer of sporting device 304 may distribute software and updates software for its systems. The application may be downloaded for free or for a certain cost, and updates may be provided also for a reduced cost.

In one example, the application 348 may receive data from sporting device 304 and generate graphical information with that data that is especially convenient and usable for an athlete to understand her performance in a sporting event. The application may also provide data to sporting device 304 such as to cause sporting device 304 to capture and preprocess data for one or more types of exercises or other operations. Data storage 346 may store raw data from sporting device 304, derived data created by electronics in sporting device 304 from the raw data, further derived data created from the other two types of data by portable device 340, video from a head-mounted device worn by the athlete, video from a head-mounted device worn by a coach, and other appropriate data that may be used for reviewing and judging the performance of an athlete in a particular group of exercises or sporting event.

Portable device 340 may serve as a sort of “master” for heads-up display 302, in that portable device 340 may perform processing on data generated by sporting device 304 and may simplify such data into a form that may be more easily handled by limited computing power of heads-up display 302. For example, heads-up display 302 may be provided only with bit-mapped data to be displayed, and may be relieved of performing the computations and layout in order to produce the bit-mapped displays.

Portable device 340 may also or alternatively serve as an adjunct to heads-up display 302, such as providing a larger and more detailed display for review of data, video, and other information by an athlete, or to permit review of the data or video by multiple people at one time. Thus, in this manner, the interrelationship of the three devices shown in FIGS. 3A and 3B in a system may permit an athlete or coach to have convenient and immediate feedback on the court simply by looking through the heads-up display 302, and to have additional ability to review information (potentially more complex and more helpful information) by pausing a practice and looking at portable device 340.

In addition, the long-range communication capabilities of portable device 340 may enable data collected at the location of an athlete to be uploaded to remote servers so as to be accessed later or combined with data from other athletes, and to obtain such data from other athletes and from other sessions by the same athlete. For example, portable device 340 may use a program to cause a query to be performed on data stored in a central server system so as to cause portable device 342 sure compared to view a performance by a particular athlete in comparison to performance by other athletes, or earlier performances by the same athlete. Therefore, the system 300 may permit ready access to a wide variety of information and powerful presentation of such information in a way that is readily understood by athletes and their coaches.

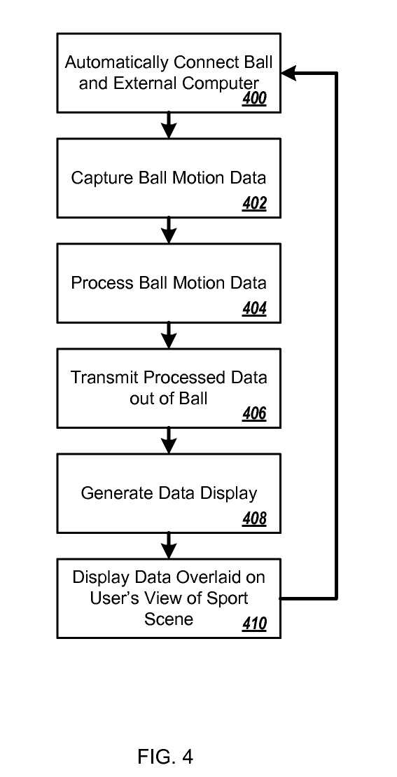

FIG. 4 is a flow chart of a process for capturing sports data and presenting it via a head-mounted device or devices. In general, the process involves capturing data that characterizes sporting activities, including capturing data about the motion of a sporting device such as a ball in the form of a basketball, volleyball, or soccer ball, and presenting the data in real-time, as it is captured, on a head-mounted display that annotates an actual view of a user with electronically-generated information generated at least in part from the data collected by the device, such as information in textual, graphical, or video form.

The process begins at box 400, where a sporting device such as a basketball or other form of ball is automatically connected to an external computer. Such connection is needed because electronics inside the ball need to be activated and to learn whether electronics are located nearby that can receive collected data. The activation may occur, for example, by bouncing the ball in a predefined pattern such as bouncing it hard against the floor three times in under a second. A mechanism in the ball may accept such action as a command to turn on the electronics within the ball and to establish a handshaking mechanism or other mechanism for seeking and identifying devices in the vicinity of the ball for wireless communication with any identified access points or networks that the electronics in the ball can currently see. Such establishment of a communication link may occur according to familiar mechanisms such as those used with WiFi and Bluetooth communications, among others.

At box 402, ball motion data is captured by electronics located inside the ball. Such electronics may include an inertial motion package that may obtain information relating to acceleration in multiple planes, or dimensions, forces applied to the ball, speed and rotation of the ball, and other similar information that is commonly collected with commercially available sensor packages of the type.

At box 404, the ball motion data is processed. Such processing may occur in a number of different steps and by way of a number of different devices. For example, initial processing may occur within the ball itself using a microprocessor or dedicated processor to turn raw motion sensor data into data that is specific to the particular sport to which the sporting. As one example, circuitry in the ball may be programmed to recognize patterns that correspond to a particular sport, such as recognizing motion data collected by the device and matching it to actions of dribbling, losing a dribble, preparing to and taking a shot, and passing the ball.

At box 406, the processed data is transmitted out of the ball, such as directly to a heads-up display located by a player handling the ball, to a different computer worn by such player (e.g., a smartphone), or to an access point from which the data may be forwarded to a server system and processed and then sent to various heads-up displays.

At box 408, a data display is generated for presentation on a heads-up device. Such generation may occur in a single step at the heads-up display or by a separate computer generating a bitmap or other image for the heads-up display. The generation may also occur in multiple steps, such as by the generation of an HTML or other form of document, and then the interpretation and rendering of such document by the heads-up display. The latter technique can permit greater flexibility and lower bandwidth usage by the system. For example, a small web page may be generated using AJAX techniques by which the score and time in a game is constantly updated on the edge of a heads-up display, and other information is continuously updated in a central portion of the display, such as by showing data derived from motion sensing in a ball immediately after some notable event occurs with the ball (e.g., a score being made), and then being removed and perhaps replaced with additional real-time data after another notable event is determined to occur.

At box 410, the data is displayed as an overlay on a user's view of a sport scene. Techniques for such display are shown and discussed above, and may include directly overlay into the center of a user's field of view through a pair of glasses, or overlay in a small portion of the field of view, such as using a small display device in a corner of the field of view.

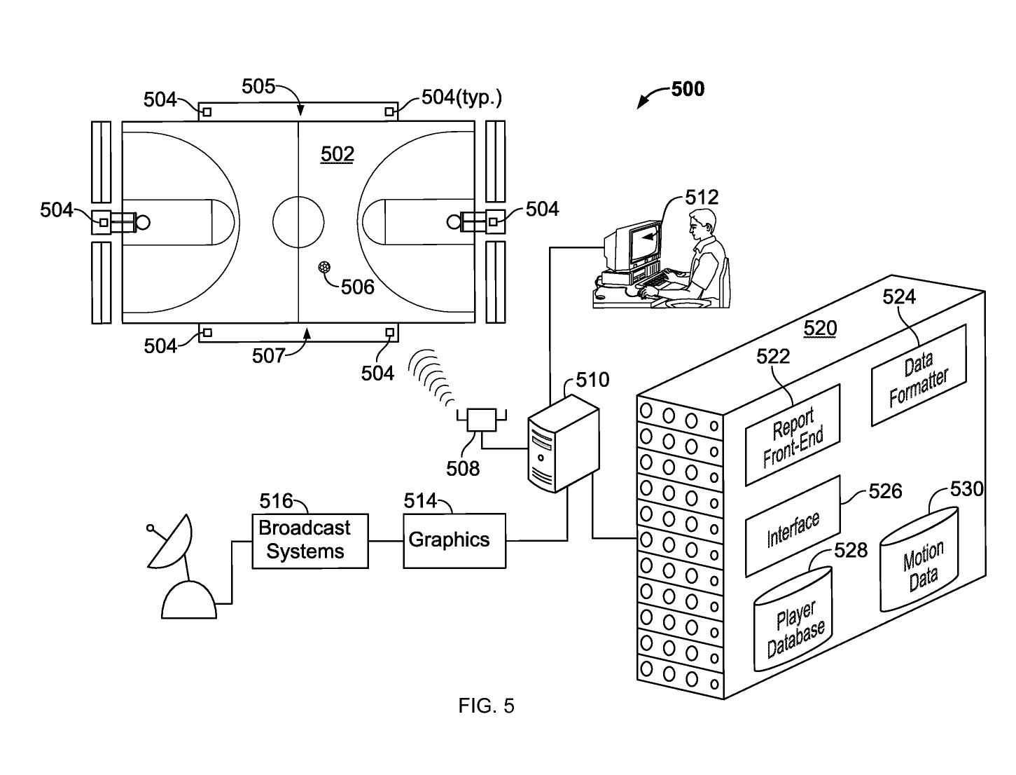

FIG. 5 is a block diagram of an illustrative system 500 for collecting, presenting, and storing data from a sporting event. In general, the system 500 is directed to capturing motion data from an athletic device that is handled by a number of different athletes during an athletic event—such as a basketball, baseball, soccer ball, and other such device—and converting the motion data for real-time visual or audible presentation along with video captured of the athletic event (and for subsequent storage and use of such data). For example, data that characterizes the actual motion of a ball can be converted into a representative number or a graph and can be super-imposed at the edge of a television screen for an ongoing game or on another device, such as on a mobile computer tablet.

Such motion data may also be time-aligned with the game clock as the data is captured, and other relevant data can likewise be aligned with the game clock, both as it is captured, and further aligned using the motion data. For example, a change of possession between two players on a basketball team can be indicated by a human analyst who is watching a basketball game, though the entry of such information will be naturally delayed somewhat from the actual time that the change of possession occurred. The motion data that has been aligned with the clock from the time such motion data is captured (with a non-appreciable delay) may then be used to identify the precise time of the change of possession (using profiles of motion that represent various predictable events such as passes, shots, alley oops, and dunks), and the analyst-entered data may be aligned with the clock at such identified times. Yet additional data may be captured, such as real-time temperature and wind data for a football game, and location information that indicates where on a court or field the ball and various players were located at various times during a game. Such information, like the motion data, may be captured automatically in real-time and may thus be naturally aligned with the game clock.

In providing such data to viewers of an event, the raw motion data may be converted into a human-understandable form. A human-understandable form is one that can be understood by a typical sports fan, such as the hang-time of a ball, a graph showing the path of a ball, the power with which a ball was hit, and similar representations (in contrast, e.g., to complex data emitted by an accelerometer, which a typical human could not understand without further processing). Generally, in the system 500, multiple values of raw data are combined into a simpler representation in order to form the human-understandable data. For example, multiple complex sensor readings may be combined to determine the number of revolutions a ball made between leaving a player's hand or foot and before making a goal, or the RPMs of the ball may be computed using a time taken from the on-ball data or from an external timer that is compared to the motion data.

The various pieces of data, and in particular, motion data that is associated with a particular player from among multiple players in a game, may also be stored for later analysis and presentation. For example, the amount of time that a particular player controls a ball in a game may be recorded after adding up each of the individual possessions for the player, where the times at which a player gained or lost possession are determined using motion sensors in the ball. Also, the speed with which a player performs certain operations with a ball may be checked, and an average for the player may be produced.

Such statistical information that is derived from the motion data, and perhaps from other data gathered outside the ball or other item that moves, may then be used in various ways. For example, an NFL analysis program may analyze the average time that particular running backs carry a ball before being tackled and a play is whistled dead, or before being first hit and after being first hit but before the whistle blows. Such a statistic may be interesting if a running back that has the longest time standing has a very low average yards per carry, or a very high average yards per carry.

With such large amounts of raw data available, machine learning techniques may also be used to identify correlations between particular measured values and actual athletic performance. For example, a system may be trained with data from motions sensors, and associated scoring data for various players. From such training, a system may identify relevant correlations that may not have been apparent from subjective player evaluation. For example, shot angle may be correlated with scoring efficiency under certain different situations, such as to identify whether particular shooting angles work better from various different directions of shot around a bucket, and certain various distances from which shots are taken.

Such data may also be made available on-demand via one or more software applications that may be correlated to video on-demand of sporting events. For example, the top N actions for a night or week of sports may be identified by a system, such as the 10 strongest dunks as measured by G force of the respective dunks. Such dunks may be displayed in a list that shows the game in which the dunk occurred, the G forces, and the name of the player who made the dunk. A user of a smartphone, tablet, or other computer may select one of the entries in the list to have video of the dunk displayed to them, and may subsequently choose to “like” or “endorse” the dunk so that a link to the video is displayed to their friends in a social network, for example.

The information produced visually by such applications may be produced for display on one or more head-mounted displays, such as GOOGLE GLASS, worn by players, coaches, studio analysts, referees/officials, or spectators. For such users, the additional data may be provided in an unobstrusive manner that does not block a natural view of a sporting event by a user, but that a user can easily look to if they want to see extra information. For example, the information can fill the user's field of view substantially but be projected onto a transparent surface so as to overly but not obstruct the natural field of view. Alternatively, or in addition, the information may be displayed in a manner that blocks only a small portion of a user's field of view, on a transparent, semi-transparent, or opaque surface.

Referring now more directly to the particular structural components in FIG. 2 that permit presentation of live game data to a wearer of a head-mounted display, an illustrative system 200 for collecting, presenting, and storing data from a sporting event is shown. In general, the system 500 includes a number of mechanisms for capturing game play data, including sensors in a basketball 506 and positioned around a court 502, and data entered by a human observer of a game. That data is telemetrically captured and stored in a database in a manner that they can be associated with other occurrences as part of the game, such as by linking all data to a timeline that is common with timing for video that displays the game play. The system 500 may represent a particular implementation of such a monitoring and analysis system as is shown with respect to FIG. 1 above and the other figures below.

In the system 500, the court 502 is shown with a ball 506 in play (though the players are not shown here, to make the image clearer). Sensors (e.g., combined package of gyroscopes, accelerometers, and magnetometers) may be located in the ball 506, including accelerometer and gyro sensors. Also in the ball 506 is a wireless transmitter and associated electronics for telemetrically sending data in real-time from the ball 506 to transceivers 504 that are positioned around the court 502. Such communication may occur according to a typical wireless standard such as Bluetooth, WiFi, or the like. Separate sensors may be located in courtside advertising boards 507 on each side of the court 502 and may be used, e.g., to identify the location of the ball 506 and/or players on the court 502, such as by using known triangulation techniques or other position determination techniques. Also, a human observer at a terminal 512 may also enter data, such as brief textual descriptions, statistics, and score changes—similar to statistics like those traditionally shown with the ESPN GameCast system (e.g., made and missed shots, fouls, etc.).

The various sensors communicate wirelessly to a router 508 that is connected to a monitoring computer system 510, which may have one or more computers programmed to convert data generated by the various sensors into alternative forms. The computers may be located at a site of the sporting event, at a remote site, or at a combination of the two.

As one example, the various forms of data (e.g., from sensors in the ball 506 and from other sources) may be time-aligned with each other and with a game clock for the basketball game and/or another running clock, so that subsequent querying for data may be used to obtain a portion of video or audio for the game, or to obtain corresponding statistics, such as to show the score of the game when a certain motion event took place, or the person who possessed the ball when the motion event took place. The common clock may then be used to pull up multiple types of such data in coordination. As one example, a user might query a database of data for a large number of games, looking for g-force data above a certain level in the last 30 seconds of a game, and in time-wise alignment of the game, look for large score changes in the game (increasing by 5 points), so as to automatically be provided with video of thunderous game-winning dunks. Such a user may be a technician at a company that provides data and video to a television network, or may also be a consumer who has downloaded an app to a smartphone or tablet computer, so that the combined and aligned data permit convenient locating of particular types of events within a very large database.

The various gathered data may be provided to a graphics system 514, which may be used to query the data, either in system 510 or in database management system 520, and may provide graphics for superposition with a television video feed associated with the game that is provided by broadcast system 516, such as through a satellite uplink for further broadcast to a local area, nationally, or worldwide. The graphics system 514 may be arranged to provide a number of different output data forms. One output form may be graphics designed to be placed over a broadcast television feed of the game as it is ongoing, such as to show a pop-up graphic about a certain player's historical performance associated with a parameter measured from the ball (e.g., time to get the ball to the floor on a first dribble when a point guard drives to a basket). Another output may be designed for presentation on heads-up displays of users watching at home and/or in the arena, such as by presenting the same data that is provided for the broadcast feed but in a different format so as to match the presentation style of the heads-up displays (e.g., showing game clock and score continuously and supplementing that data with temporary statistics as text or graphics). The data for the heads-up displays may be formatted as HTML, CSS, JavaScript, XML or similar data rather than just a video or image feed, and the heads-up displays may determine how to format such information for display according to familiar techniques.

The database management system 520 may be a central system remote from the game that stores motion data from a large number of games, perhaps for an entire league and for multiple different sports, and may be a system operated by a service bureau that provides third party access to data, such as motion data of game balls, to subscribers that can include television networks. Local processing at the event may be used to generate graphic overlays for real-time or near real-time television broadcast, whereas processing remote from the event may occur for less time-sensitive and less specific uses, such as for access by members of the public, or for research by computer technicians looking for statistics to display with an analysis program on the network.

Certain components are shown as example structural components that the database management system 520 can use to provide such information. For example, a report front-end 522, which may be in the form of a web server or similar interface, can be used to receive query parameters from a user or an automated data extraction system and can provide a user interface for manual requests (e.g., in the form of JavaScript, HTML, or XML code that can be served to a large number of remote client computing devices). The font-end 522 may parse received requests and convert them to an appropriate query (e.g., SQL) to be applied to a motion data 530 database that contains different forms of motion data, including data gathered by in-ball sensors. The other data may be part of the same database system 520 or part of a separate system, including a separate organizational entity with which the operator of system 520 has a data sharing agreement, wherein the communication occurs according to previously agreed-upon application programming interfaces (APIs).

As one example, a player database 528 may store data about particular players, including traditional statistics (e.g., shots made and missed, points per game, minutes played, rebounds, etc.). Additionally, the player database 528 (either in a common database or in databases split across multiple systems) may store motion-related data about a player, either in raw form or in a derived form. The raw form may include particular accelerometer data and other motion data over time periods during which the player was handling a basketball. The derived data may include, for example, numbers that represent the maximum dribbling force at the beginning of each scoring drive by the player. The decision whether to employ raw data versus derived data may depend on the fact that the former is more detailed but is also more difficult and time-consuming to query or otherwise process—with the decision in each particular implementation depending on a particular balancing of factors.

A data formatter may interact with located search results from the databases and provided output for presentation via interface 526. For example, the data formatter 524 may generate a table or graph from information, and interface 526 may serve such a presentation, including by serving it in response to a technician at a statistical analysis company and/or an operator at a television broadcasting system. For example, a television technician may recognize that a color commenter at a basketball game has commented several times about a center's speed in picking up the dribble and shooting. The technician may then remotely query the system 520, identifying particular events associated with picking up a dribble and shooting, in order to obtain an average velocity profile for shots made when the center is under the basket (i.e., standing lay-ups or dunks), and can identify five other centers with whom the data is to be compared. The system 520 may obtain such data, and the data formatter 524 may form graphs that show the paths (e.g., as viewed from the side) of each player raising the ball from a dribble to a shot, and may color each portion of each path in a color that indicates each player's relative speed at that point along the path. As a result, the commenter may immediately illustrate the point he has been making throughout the game, and his expertise as an analyst may be backed up with the real motion data. Of course, more complex and specific analyses and graphics may be prepared in advance of a game and can be shown at an appropriate time, including with updated information from the current ongoing game. Such graphics may also be provided in the same format or a different format to heads-up displays worn by spectators.

In this manner then, the system 500 may collect various forms of raw data—including from sensors in the ball or other playing item that is handled by players, from human observers of a game, and from sensors outside the ball or other handled item—and may store the data and make it available for various forms of subsequent analysis and display in a combined and correlated (e.g., time-aligned) manner. Such analysis may be predetermined, where the data is fed into predefined analysis mechanisms and automatically fed to a predefined on-screen display (e.g., to display the force of a dunk immediately as the dunk is made or immediately after, either fully automatically or in response to a broadcast technician making a simple selection on a control computer to have such information displayed).

The particular techniques described here may be assisted by the use of one or more computers, such as wearable computers (e.g., in the form of glasses with a heads-up display), servers, desktops, smartphones, and tablets. The computing portions of such devices are shown generally in FIG. 6, and may communicate with and/or incorporate a computer system 600 in performing the operations discussed above, including obtaining and processing incoming motion data, and formatting and presenting information from such data in a tabular or graphical manner that is pleasing and useful to a view with a head-mounted display.

The system 600 may be implemented in various forms of digital computers, including computerized laptops, personal digital assistants, tablets, and other appropriate computers. Additionally the system can include portable storage media, such as, Universal Serial Bus (USB) flash drives. For example, the USB flash drives may store operating systems and other applications. The USB flash drives can include input/output components, such as a wireless transmitter or USB connector that may be inserted into a USB port of another computing device.

The system 600 includes a processor 610, a memory 620, a storage device 630, and an input/output device 640. Each of the components 610, 620, 630, and 640 are interconnected using a system bus 650. The processor 610 is capable of processing instructions for execution within the system 600. The processor may be designed using any of a number of architectures. For example, the processor 610 may be a CISC (Complex Instruction Set Computers) processor, a RISC (Reduced Instruction Set Computer) processor, or a MISC (Minimal Instruction Set Computer) processor.

In one implementation, the processor 610 is a single-threaded processor. In another implementation, the processor 610 is a multi-threaded processor. The processor 610 is capable of processing instructions stored in the memory 620 or on the storage device 630 to display graphical information for a user interface on the input/output device 640.

The memory 620 stores information within the system 600. In one implementation, the memory 620 is a computer-readable medium. In one implementation, the memory 620 is a volatile memory unit. In another implementation, the memory 620 is a non-volatile memory unit.

The storage device 630 is capable of providing mass storage for the system 600. In one implementation, the storage device 630 is a computer-readable medium. In various different implementations, the storage device 630 may be a floppy disk device, a hard disk device, an optical disk device, or a tape device.

The input/output device 640 provides input/output operations for the system 600. In one implementation, the input/output device 640 includes a keyboard and/or pointing device. In another implementation, the input/output device 640 includes a display unit for displaying graphical user interfaces.

The features described can be implemented in digital electronic circuitry, or in computer hardware, firmware, software, or in combinations of them. The apparatus can be implemented in a computer program product tangibly embodied in an information carrier, e.g., in a machine-readable storage device for execution by a programmable processor; and method steps can be performed by a programmable processor executing a program of instructions to perform functions of the described implementations by operating on input data and generating output. The described features can be implemented advantageously in one or more computer programs that are executable on a programmable system including at least one programmable processor coupled to receive data and instructions from, and to transmit data and instructions to, a data storage system, at least one input device, and at least one output device. A computer program is a set of instructions that can be used, directly or indirectly, in a computer to perform a certain activity or bring about a certain result. A computer program can be written in any form of programming language, including compiled or interpreted languages, and it can be deployed in any form, including as a stand-alone program or as a module, component, subroutine, or other unit suitable for use in a computing environment.

Suitable processors for the execution of a program of instructions include, by way of example, both general and special purpose microprocessors, and the sole processor or one of multiple processors of any kind of computer. Generally, a processor will receive instructions and data from a read-only memory or a random access memory or both. The essential elements of a computer are a processor for executing instructions and one or more memories for storing instructions and data. Generally, a computer will also include, or be operatively coupled to communicate with, one or more mass storage devices for storing data files; such devices include magnetic disks, such as internal hard disks and removable disks; magneto-optical disks; and optical disks. Storage devices suitable for tangibly embodying computer program instructions and data include all forms of non-volatile memory, including by way of example semiconductor memory devices, such as EPROM, EEPROM, and flash memory devices; magnetic disks such as internal hard disks and removable disks; magneto-optical disks; and CD-ROM and DVD-ROM disks. The processor and the memory can be supplemented by, or incorporated in, ASICs (application-specific integrated circuits).

To provide for interaction with a user, the features can be implemented on a computer having an LCD (liquid crystal display) or LED display for displaying information to the user and a keyboard and a pointing device such as a mouse or a trackball by which the user can provide input to the computer.

The features can be implemented in a computer system that includes a back-end component, such as a data server, or that includes a middleware component, such as an application server or an Internet server, or that includes a front-end component, such as a client computer having a graphical user interface or an Internet browser, or any combination of them. The components of the system can be connected by any form or medium of digital data communication such as a communication network. Examples of communication networks include a local area network (“LAN”), a wide area network (“WAN”), peer-to-peer networks (having ad-hoc or static members), grid computing infrastructures, and the Internet.

The computer system can include clients and servers. A client and server are generally remote from each other and typically interact through a network, such as the described one. The relationship of client and server arises by virtue of computer programs running on the respective computers and having a client-server relationship to each other.

Many other implementations other than those described may be employed, and may be encompassed by the following claims.

What is claimed is:

1. A computer-implemented method comprising:
capturing data about motion of a sports object caused by one or more athletes manipulating the sports object;
transmitting the captured data out of the sports object wirelessly in real time while the one or more athletes are still manipulating the sports object; and
presenting information that incorporates the captured data about motion of the sports objects with one or more wearable devices.

2. The computer-implemented method of claim 1, wherein the sports object is a sports ball and the data is capturing by one or more motion sensors mounted inside the sports ball.

3. The computer-implemented method of claim 2, wherein the wearable devices are worn by the one or more athletes and the presented information provides them with feedback about their athletic performance with the sports ball.

4. The computer-implemented method of claim 3, wherein the wearable devices comprise electronic glasses having a visual display that shows the presented information.

5. The computer-implemented method of claim 4, wherein the electronic glasses are connected to a computer and wireless receiver arranged to obtain data from a wireless transmitter inside the sports ball in real-time.

6. The computer-implemented method of claim 5, wherein the electronic glasses present to an athlete currently manipulating the sports ball a numerical indication of their manipulation of the sports ball.

7. The computer-implemented method of claim 5, further comprising automatically wirelessly connecting the wireless receiver and wireless transmitter upon sensing that the wireless receiver and wireless transmitter are near each other.

8. The computer-implemented method of claim 5, further comprising recording a video that comprises video captured by the electronic glasses overlaid with information derived from the captured data about motion of the sports ball.

9. The computer-implemented method of claim 2, further comprising determining whether the sports ball is undergoing dribbling actions or is undergoing shooting actions, and selecting a type of information to be displayed based on the determination, wherein:
If the sports ball is determined to be undergoing dribbling actions, the presented information indicates a quality of the dribbling across multiple dribbles; and
If the sports ball is determined to be undergoing shooting action, the presented information comprises indications of arc, accuracy, or both for a current shot.

10. The computer-implemented method of claim 1, wherein the wearable devices comprise electronic glasses worn by a plurality of spectators watching the plurality of athletes in person, and the presented information annotates action in a game that the plurality of athletes are currently playing.

11. The computer-implemented method of claim 1, further comprising recognizing a verbal command spoken by one of the one or more athletes, and determining a mechanism to use in analyzing data from the sports item in response to content of the verbal command.

12. The computer-implemented method of claim 1, further comprising communicating between worn computing devices of different ones of the one or more athletes to share information from the sports device.

13. The computer-implemented method of claim 1, further comprising presenting with the wearable devices and to one of the one or more athletes, instructions for improving athletic performance for the one of the one or more athletes, using the captured data.

14. A computer-implemented system comprising:
a wearable computing device having a head-mounted display that is capable of superimposing a display of data over a field of view of a user wearing the head-mounted display;
one or more programs on one or more tangible recordable media of the wearable computing device having instructions that when executed, perform operations including:
receiving data about motion of a an athlete-manipulated sports object transmitted from a computing device inside the athlete-manipulated sports object, and
presenting information via the head-mounted display information that incorporates the captured data about motion of the athlete-manipulated sports object.

15. The computer-implemented system of claim 14, wherein the sports object is a sports ball and the data is capturing by one or more motion sensors mounted inside the sports ball.

16. The computer-implemented system of claim 15, wherein the wearable devices computing device is arranged to be worn an athlete and the presented information provides the athlete with feedback about his or her athletic performance with the sports ball.

17. The computer-implemented system of claim 14, wherein the wearable computing device comprises electronic glasses having a visual display that shows the presented information.

18. The computer-implemented system of claim 17, wherein the electronic glasses are connected to a computer and wireless receiver arranged to obtain data from a wireless transmitter inside the sports object in real-time as the data is transmitted form the sports object.

19. The computer-implemented system of claim 18, wherein the electronic glasses present, to an athlete currently manipulating the sports ball, a numerical indication of their manipulation of the sports ball.

20. The computer-implemented system of claim 18, wherein the wearable computing device and sports object are further arranged to automatically wirelessly connect the wireless receiver and wireless transmitter upon sensing that the wireless receiver and wireless transmitter are near each other.

21. The computer-implemented system of claim 17, wherein the wearable computer is further arranged to record an electronic video that comprises video captured by the electronic glasses overlaid with information derived from the captured data about motion of the sports ball.

22. The computer-implemented system of claim 14, wherein the system is further arranged to determine whether the sports object is undergoing dribbling actions or is undergoing shooting actions, and select a type of information to be displayed based on the determination, wherein:
If the sports ball is determined to be undergoing dribbling actions, the presented information indicates a quality of the dribbling across multiple dribbles; and
If the sports ball is determined to be undergoing shooting action, the presented information comprises indications of arc, accuracy, or both for a current shot.

23. The computer-implemented system of claim 14, wherein the wearable device comprises electronic glasses worn by a plurality of spectators watching the plurality of athletes in person, and the presented information annotates action in a game that the plurality of athletes are currently playing.

24. The computer-implemented system of claim 14, wherein the wearable computing device is further arranged to recognize a verbal command spoken by one of the one or more athletes, and determine a mechanism to use in analyzing data from the sports object in response to content of the verbal command.

25. The computer-implemented system of claim 14, wherein the system is further arranged to communicate between wearable computing device and a wearable computing device of one or more other athletes to share information from the sports object.

26. The computer-implemented system of claim 1, wherein the system is further arranged to present with the wearable computing device and other wearable computing and to one of the one or more athletes, instructions for improving athletic performance for the one of the one or more athletes, using the captured data.
